(19) United States
(12) Patent Application Publication
(10) Pub. No.: US 2006/0286067 A1
(43) Pub. Date: Dec. 21, 2006

(54) Methods for making and using regulatory T cells

(75) Inventors: David A. Horwitz, Santa Monica, CA (US); John Dixon Gray, Los Angeles, CA (US)

Correspondence Address: Richard F. Trecartin;Dorsey & Whitney LLP, Intellectual Property Department, 555 California Street, Suite 1000, San Francisco, CA, 94104-1513, US

(73) Assignee: University of Southern California

(21) Appl. No.: 11/400,950

(22) Filed: Apr. 5, 2006

Related U.S. Application Data
(60) Provisional application No. 60/668,676, filed on Apr. 5, 2005.

(51) Int. Cl. A61K 35/14; A61K 39/395; C12N 5/08

(52) U.S. Cl. 424/85.1; 424/144.1; 424/93.7; 435/372

(57) ABSTRACT

The invention is generally related to methods of making regulatory T cells and treating autoimmune diseases, including both antibody-mediated and cell-mediated disorders.

### PRIORITY CLAIM

This application claims priority to, and the benefit of, under 35 U.S.C. §119(e), U.S. Provisional Application Ser. No. 60/668,676, filed Apr. 5, 2005, which is incorporated in its entirety by reference herein.

### FIELD OF THE INVENTION

The field of the invention is generally related to methods for making regulatory T cells (T regs) and treating autoimmune diseases with said T regs.

### BACKGROUND OF THE INVENTION

Autoimmune diseases are caused by the failure of the immune system to distinguish self from non-self. In these diseases, the immune system reacts against self tissues and this response ultimately causes inflammation and tissue injury. Autoimmune diseases can be classified into two basic categories: antibody-mediated diseases such as systemic lupus erythematosus (SLE), pemphigus vulgaris, myasthenia gravis, hemolytic anemia, thrombocytopenia purpura, Grave's disease, Sjogren's disease and dermatomyositis; and cell-mediated diseases such as Hashimoto's disease, polymyositis, disease inflammatory bowel disease, multiple sclerosis, diabetes mellitus, rheumatoid arthritis, and scleroderma.

In many autoimmune diseases, tissue injury is caused by the production of antibodies to native tissue. These antibodies are called autoantibodies, in that they are produced by a mammal and have binding sites to the mammal's own tissue. Some of these disorders have characteristic waxing and waning of the amount of circulating autoantibodies causing varying symptoms over time.

Of the different types of antibody-mediated autoimmune disorders, SLE is a disorder that has been well studied and documented. SLE is a disorder of generalized autoimmunity characterized by B cell hyperactivity with numerous autoantibodies against nuclear, cytoplasmic and cell surface antigens. This autoimmune disease has a multifactorial pathogenesis with genetic and environmental precipitating factors (reviewed in Hahn, B. H., Dubois' Lupus Erythematosus, 5th Ed. (1997), pp. 69-76 (D. J. Wallace et al. eds., Williams and Wilkins, Baltimore)). Among the numerous lymphocyte defects described in SLE is a failure of regulatory T cells to inhibit B cell function (Horwitz, D. A., Dubois' Lupus Erythematosus, 5th Ed. (1997), pp. 155-194 (D. J. Wallace et al. eds., Williams and Wilkins, Baltimore)). Sustained production of polyclonal IgG and autoantibodies in vitro requires T cell help (Shivakumar, S. et al. (1989), J Immunol 143:103-112).

Circulating B lymphocytes spontaneously secreting antibodies are increased in patients with active SLE (Klinman, D. M. et al. (1991), Arthritis Rheum 34:1404-1410). Clinical manifestations of SLE include a rash (especially on the face in a “butterfly” distribution), glomerulonephritis, pleurisy, pericarditis and central nervous system involvement. Most patients are women, and are relatively young (average age at diagnosis is 29).

Regulatory T cells can down-regulate antibody synthesis by contact-dependent or cytokine-mediated mechanisms. The latter involve transforming growth factor-beta (TGF-β) and other inhibitory cytokines (Wahl, S. M. (1994), J Exp Med 180:1587-190. (Horwitz D A., et al., J Leukoc Biol. 2003 October;74(4):471-8). The forkhead transcription factor, FoxP3 has a critical role in the development of CD4+ cells that constitutively express the alpha chain of the IL-2 receptor CD25 (Ziegler S. F., Annu Rev Immunol. 2006 24:209-26), and also for CD8+ Tregs (Maggi E, et al., Autoimmun Rev. 2005 November;4(8):579-86). Natural thymus derived CD4+CD25+ cells express FoxP3, and acquired CD4+CD25+ Tregs derived from peripheral CD4+CD25− precursors express FoxP3. Stable expression of FoxP3, therefore, can serve as a marker of specific subsets of Tregs.

The treatment of SLE depends on the clinical manifestations. Some patients with mild clinical symptoms respond to simple measures such as nonsteroidal anti-inflammatory agents. However, more severe symptoms usually require steroids with potent anti-inflammatory and immunosuppressive action such as prednisone. Other strong immunosuppressive drugs which can be used are azathioprine and cyclophosphamide. The steroids and other immunosuppressive drugs have side effects due to the global reduction of the mammal's immune system. There is presently no ideal treatment for SLE and the disease cannot be cured.

Currently, considerable attention has been focused on the identity of genes which enhance the susceptibility or resistance to SLE, the identification of antigenic determinants that trigger the disease, the molecular mechanisms of T cell activation which results in survival or apoptosis, cytokines which determine T cell function, and the properties of the autoantibody-forming B cells. Many examples of T cell dysregulation in SLE have been described (reviewed in Horwitz, D. A. et al., Dubois' Lupus Erythematosus, 5th Ed. (1997), pp. 83-96 (D. J. Wallace et al. eds., Williams and Wilkins, Baltimore). Although it is well recognized that the primary role of certain lymphocytes is to down-regulate immune responses, progress in elucidating the identity and mechanisms required for generation of these cells has been slow.

Interleukin-2 (IL-2) has previously been considered to have an important role in the generation of antigen non-specific T suppressor cells (Setoguchi R, et al., J Exp Med. 2005 Mar. 7;201(5):723-35.)

Anti-IL-2 antibodies given to mice coincident with the induction of graft-versus-host-disease resulted in several features of SLE (Via, C. S. et al. (1993), International Immunol. 5:565-572). Whether IL-2 directly or indirectly is important in the generation of suppression has been controversial (Fast, L. D. (1992), J Immunol. 149:1510-1515; Hirohata, S. et al. (1989), J Immunol. 142:3104-3112; Baylor, C. E. (1992), Advances Exp. Med. Biol. 319:125-135). Recently, IL-2 has been shown to induce CD8+ cells to suppress HIV replication in CD4+ T cells by a non-lytic mechanism. This effect is cytokine mediated, but the specific cytokine has not been identified (Kinter, A. L. et al. Proc. Natl. Acad. Sci. USA 92:10985-10989; Barker, T. D. et al. (1996), J Immunol. 156:4478-4483). T cell production of IL-2 is decreased in SLE (Horwitz, D. A. et al. (1997), Dubois' Lupus Erythematosus, 5th Ed. (1997), pp. 83-96, D. J. Wallace et al. eds., Williams and Wilkins, Baltimore).

CD8+ T cells from subjects with SLE sustain rather than suppress polyclonal IgG production (Linker-Israeli, M. et al. (1990), Arthritis Rheum. 33:1216-1225). CD8+ T cells from healthy donors can be stimulated to enhance antibody production (Takahashi, T. et al. (1991), Clin. Immunol. Immunopath. 58:352-365). However, neither IL-2 nor CD4+ T cells, by themselves, were found to induce CD8+ T cells to develop strong suppressive activity. When NK cells were included in the cultures, strong suppressive activity appeared (Gray, J. D. et al. (1994) J Exp. Med. 180:1937-1942). It is believed that the contribution of NK cells in the culture was to produce transforming growth factor beta (TGF-β) in its active form. It was then discovered that non-immunosuppressive (2-10 pg/ml) concentrations of this cytokine served as a co-factor for the generation of strong suppressive effects on IgG and IgM production (Gray, J. D. et al. (1994) J Exp. Med. 180:1937-1942). In addition, it is believed that NK cells are the principal source of TGF-β in unstimulated lymphocytes (Gray, J. D. et al. (1998), J Immunol. 160:2248-2254).

TGF-βs are a multifunctional family of cytokines important in tissue repair, inflammation and immunoregulation (Massague, J. (1980), Ann. Rev. Cell Biol. 6:597). TGF-β is unlike most other cytokines in that the protein released is biologically inactive and unable to bind to specific receptors (Sporn, M.B. et al. (1987) J Cell Biol. 105:1039-1045). The latent complex is cleaved extracelluarly to release active cytokine as discussed below. The response to TGF-β requires the interaction of two surface receptors (TGF-β-R1) and TGF-β-R2) which are ubiquitously found on mononuclear cells (Massague, J. (1992), Cell 69:1067-1070). Thus, the conversion of latent to active TGF-β is the critical step which determines the biological effects of this cytokine.

It was found that SLE patients have decreased lymphocyte production of TGF-β1. Defects in constitutive TGF-β produced by NK cells, as well induced TGF-β were documented in a study of 38 SLE patients (Ohtsuka, K. et al. (1998), J Immunol. 160:2539-2545). Neither addition of recombinant IL-2 or TNF-alpha, or antagonism of IL-10 normalized the TGF-β defect in SLE. Decreased production of TGF-β in SLE did not correlate with activity of disease and, therefore, may be a primary defect.

Systemic administration of TGF-β, IL-2, or a combination of both can lead to serious side effects. These cytokines have numerous effects on different body tissues and are not very safe to deliver to a patient systemically.

### SUMMARY OF THE INVENTION

Previously, regulatory T cells (T regs) were obtained by continuous culture of peripheral blood mononuclear cells (PBMC) with TGF-β, a cytokine and optionally a mitogen. Such culturing was for approximately 6-7 days and such populations could be expanded by repeated culturing in the presence of TGF-β and IL-10. When so treated, a polyclonal population of T regs is obtained. See, e.g., U.S. Pat. No. 6,797,267 B2 incorporated herein by reference. When cultured with an antigen source such as irradiated donor cells for solid organ transplantation, an antigen specific population is formed. See, e.g., U.S. 2002/0006392 A1, published Jan. 17, 2002, incorporated herein by reference.

The present invention provides improved methods for generating T regs. instead of using a continuous culture with TGF-β and a cytokine, the present invention provides a two-stage culturing of PBMCs.

In one approach, PBMCs are first cultured with a first regulatory composition comprising TGF-β and optionally a mitogen and/or cytokine such as IL-2, IL-7, IL-10 and/or IL-15. After initial culturing, the first regulatory composition is removed from the PBMCs. The PBMCs are thereafter cultured with a second regulatory composition comprising a cytokine such as IL-2, IL-7, IL10 and/or IL-15.

In a second approach, PBMCs are cultured with a first regulatory composition comprising TGF-β and optionally a mitogen and/or cytokine such as IL-2, IL-7, IL-10 and/or IL-15 to form a first culture. The first culture is then diluted with nutrient media to form a second culture.

In general, the culturing with the first regulatory composition is for 24-48 hours and the culturing with the second composition or nutrient media is for 4-6 days. This is sometimes abbreviated as a “2+4 protocol.”

The mitogen is preferably selected from a group consisting of anti-CD2, anti-CD3, anti-CD28 and combinations thereof. A particularly preferred mitogen is a combination of anti-CD3 and anti-CD28 antibodies, especially beads coated with these antibodies.

After isolating PBMCs from an individual, they can be treated to enrich for specific populations of cells including CD4, CD8 and/or NK-T cells.

The invention is also directed to regulatory T cells (Tregs) made according to the methods of the invention. As indicated herein, treatment of PBMCs according to the methods of the invention results in a population of Tregs where the number of Fox P3+ cells as a percentage of CD25+ cells is greater than that obtained by continuous culture as compared to a 2+4 protocol.

The invention also includes methods for treating autoimmune disorders by introducing the aforementioned T regs into a patient afflicted with a autoimmune disorder so as to ameliorate at least one autoimmune symptom.

### BRIEF DESCRIPTION OF THE DRAWINGS

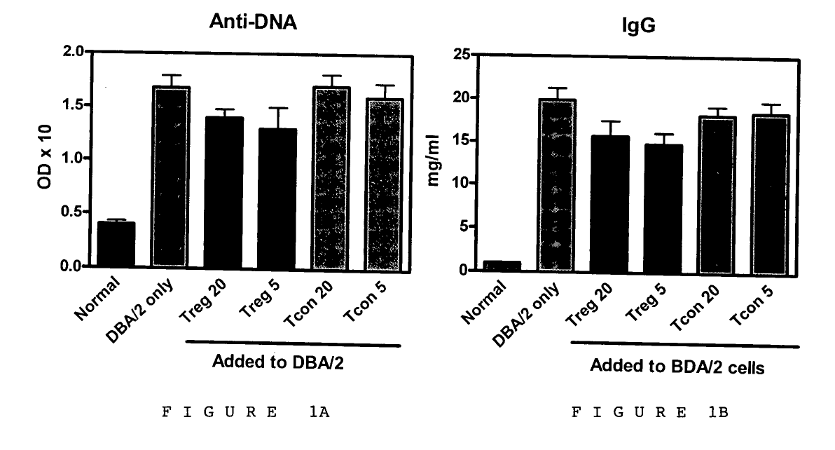

In FIGS. 1A and 1B, purified T cells from DBA/2 mice were stimulated with beads coated with anti-CD3 and anti-CD28 with IL-2 and TGF-beta1 2 ng/ml (Treg) or without TGF-beta1(Tcon) for 6 days. To induce a lupus-like syndrome, 80 million D2 spleen cells were transferred to DBA/2 × C57BL/6 F1 mice. Some groups also received 5 to 20 million Tcon or Treg (n=3). Two weeks later, the mice were bleed and assessed for anti-dsDNA antibody (FIG. 1A) and total IgG (FIG. 1B) that were measured by standard ELISA methods

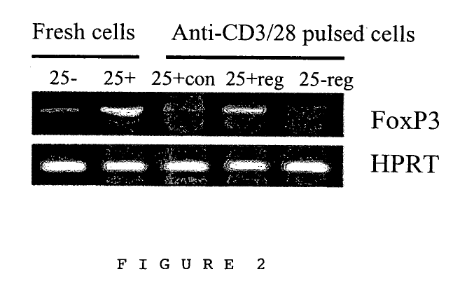

In FIG. 2, purified T cells from DBA/2 mice were stimulated with beads coated with anti-CD3 and anti-CD28 with IL-2 and TGF-beta1 2 ng/ml, (Treg) or without TGF-beta1 (Tcon) for 2 days. The beads were then removed and the cells cultured for other 4 days in the presence of IL-2 (10 u/ml). The CD4+CD25+ cells or CD4+CD25− cells were sorted from the Treg or Tcon cells and the FoxP3 mRNA expression was determined on various cell subsets by RT-PCR.

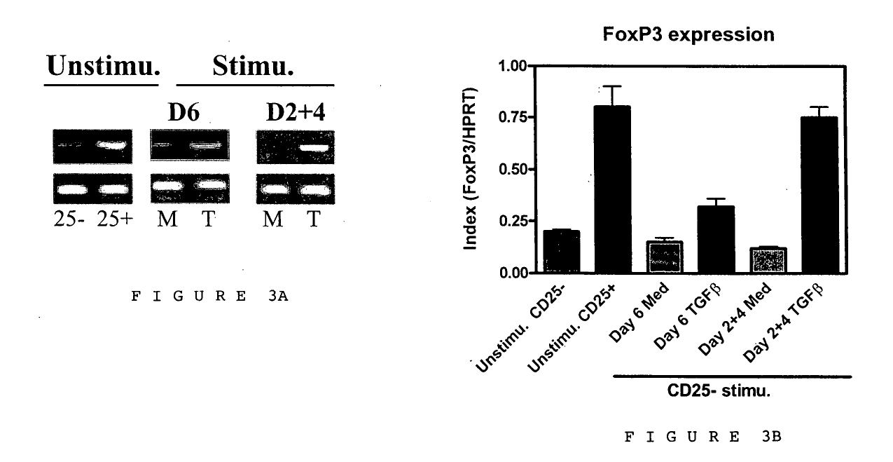

In FIGS. 3A and 3B, CD4+CD25− cells were stimulated with anti-CD3/CD28 coated beads (1:3)+IL-2 (40 u/ml) for 6 days +/−TGF-β (2 ng/ml). In some groups, these cells were stimulated with similar beads for 2 days, then beads were removed and resting for other 4 days with 10 u/ml IL-2. FoxP3 mRNA was analyzed by RT-PCR (FIG. 3A) or semiquantitative levels were determined by normalization to HPRT of three separate experiments (FIG. 3B). M indicates medium only, T indicates TGF-β.

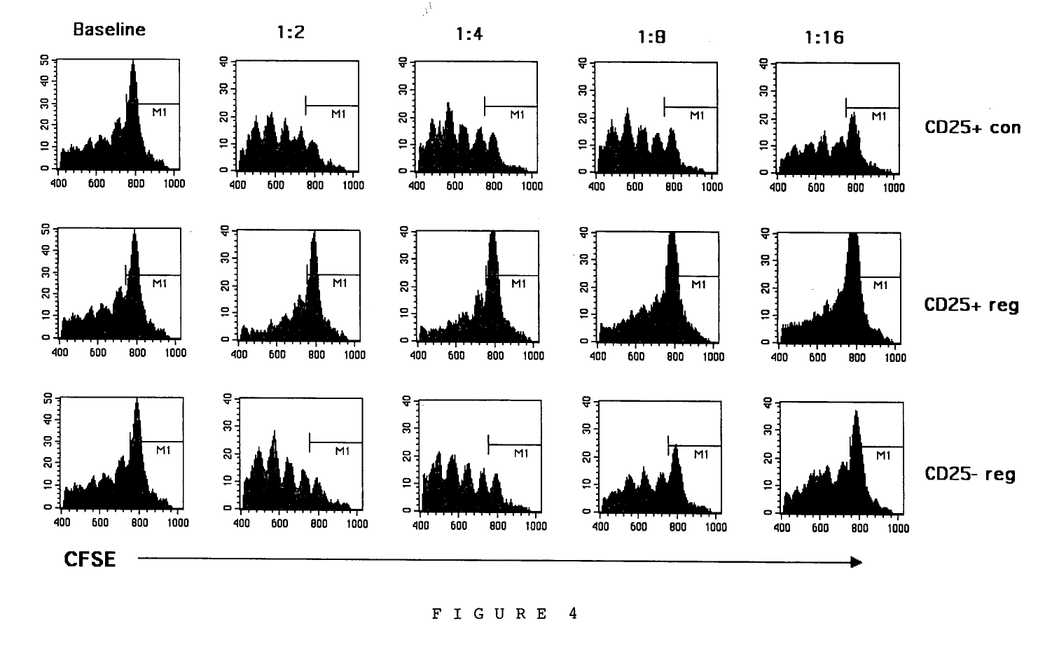

In FIG. 4, T cells were labeled with CFSE and stimulated with souble anti-CD3 in the presence of APC for 4 days. CD4+CD25+ cells were sorted and various ratios of these cells were added to culture. The CD4+ cell proliferation was analyzed by the dilution of CFSE.

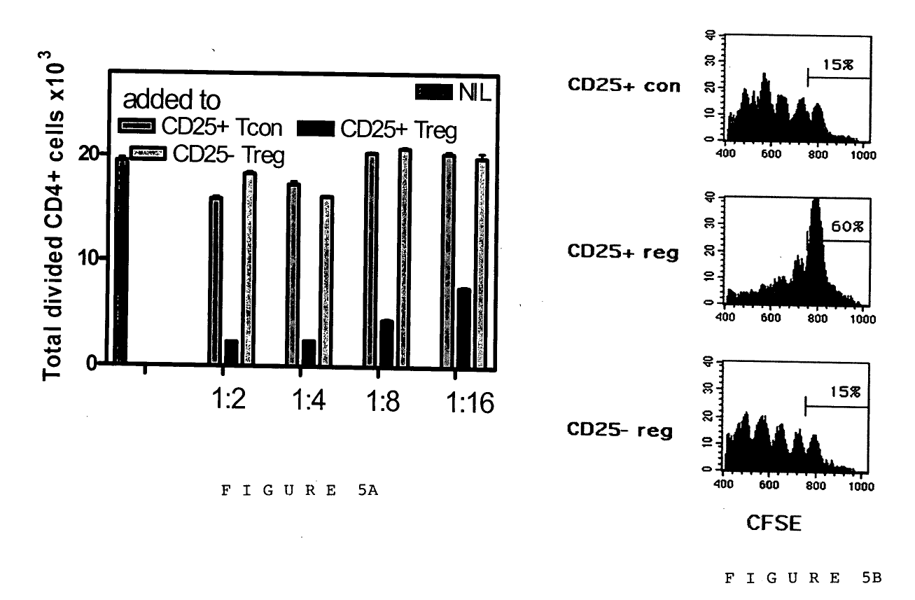

In FIG. 5, CD4+ cells that were briefly activated with a polyclonal mitogen and TGF-β and cultured for more 4 days became CD25+ cells with marked suppressive activity. Total suppression was calculated by proliferative rates multiply total cell numbers (FIG. 5A) and typical proliferative rate at 1:4 ratio was shown in FIG. 5B.

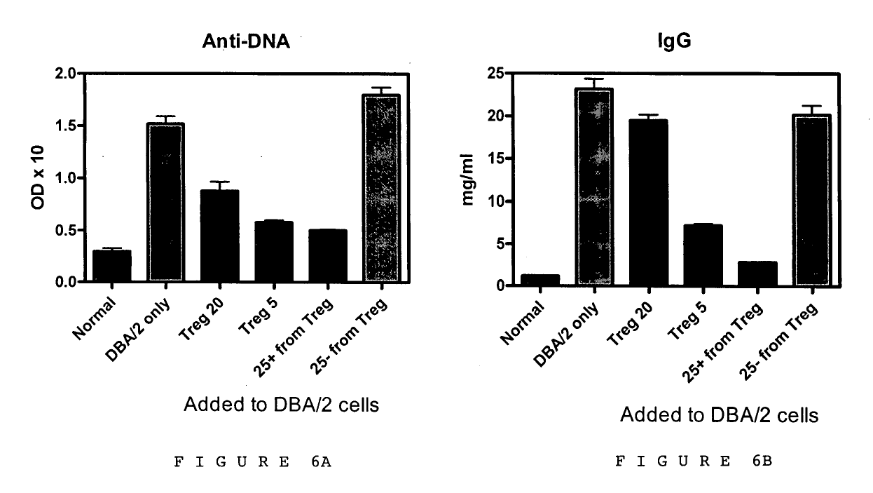

In FIGS. 6A and 6B, purified T cells from DBA/2 mice were stimulated with beads coated with anti-CD3 and anti-CD28 with IL-2 and TGF-beta1 2 ng/ml (Treg) or without (Tcon) for 2 days and remove beads and cultured for 4 days. To induce a lupus-like syndrome, 80 million D2 spleen cells were transferred to DBA/2 × C57BL/6 F1 mice. Some groups also received 5 to 20 million Tcon or Treg (n=3). Two weeks later, the mice were bled and assessed for anti-dsDNA antibody (FIG. 6A) and total IgG (FIG. 6B) that were measured by standard ELISA methods.

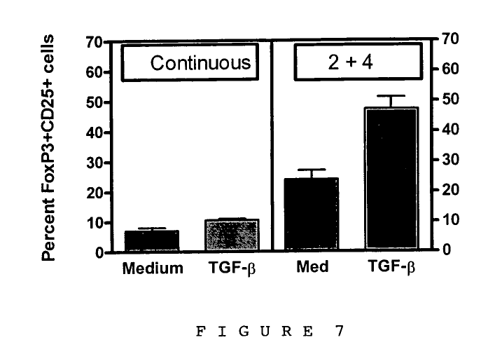

In FIG. 7, naive human CD4+ cells were stimulated with anti-CD3/CD28 beads in the presence or absence of TGF beta. The cells in some wells were cultured for 6 days (continuous). At day 2 of culture, the cells from some wells were removed, distributed into two wells, and fresh medium added to make a final volume of 1 ml. These were recultured for 4 additional days (2+4). At this time the cells were assayed for expression of FoxP3. Data represent FoxP3+ cells as a percentage of CD25+ cells.

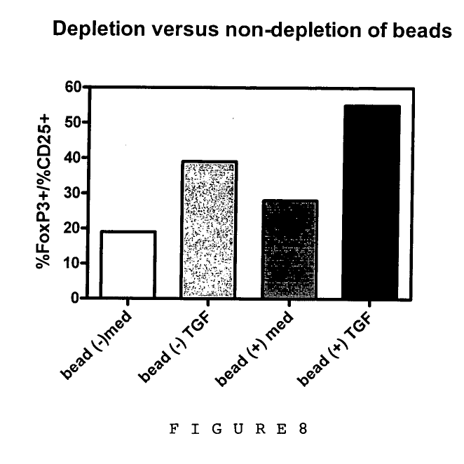

In FIG. 8, naive CD4+ cells were stimulated with anti-CD3/CD28 beads in the presence or absence of TGF beta. At day 2 of culture, the cells were removed from the wells, and split into two portions. The magnetic beads were removed from one portion. Each portion was added to new wells with additional medium to a final volume of 1 ml and the cells were recultured for 4 additional days. At this time the cells were assayed for expression of FoxP3. Data represent the % FoxP3+ cells as a percentage of CD25+ cells.

### DETAILED DESCRIPTION OF THE INVENTION

The present invention is directed to methods of making regulatory T cells and of treating autoimmune disorders, including both cell-mediated and antibody-mediated disorders such as systemic lupus erythematosus (SLE). The methods involve (1) removing PBMCs from a patent and treating them with a first regulatory composition comprising TGF-β and optionally a mitogen and/or cytokine such as IL-2, IL-7, IL-10 and/or IL-15 for 24-48 hours, (2) removing the first regulatory composition followed by (3) culturing the cells with (a) a second regulatory composition comprising a cytokine such as IL-2, IL-7, IL-10 and/or IL-15 or (b) nutrient medium for 4-6 days.

In another embodiment, the method involves (1) removing PBMCs from a patent and treating them for 24-48 hours with a first regulatory composition comprising TGF-β and optionally a mitogen and/or cytokine such as IL-2, IL-7, IL-10 and/or IL-15 to form a first culture and (2) diluting the first culture with nutrient media to form a second culture that is cultured for 4-6 days. The nutrient media can be substantially free of TGFβ and/or cytokine (i.e., neither TGFβ and/or cytokine have been added to the cell culture medium) or may contain a cytokine such as IL-2, IL-7, IL-10 and/or IL-15 with IL-2 being preferred.

TGFβ is usually present in an amount between about 1-1000 ng/ml. The cytokine is usually present in an amount from 0.1-100 IU/ml. T regs produced by such methods produce a higher number of suppressor cells as compared to continuous treatment with TGF-β and cytokine for 5-6 days.

In some embodiments, the first culture is diluted with about an equal volume of nutrient medium. In others, the first culture is divided into two or more portions which are then diluted with nutrient media. The advantage of division is that the cell clusters formed in the first culture (thousands of cells) are mechanically disrupted and form smaller cell clusters (tens to hundreds of cells) when pipetted during division of the first culture. These small clusters are then able to grow into larger clusters during the next 4-6 day culture.

The regulatory T cells are used to treat cell-mediated autoimmune disease. In this embodiment, the compositions induce immune cells to generate suppressor T cells. These suppressor T cells prevent other T cells from becoming cytotoxic and attacking the cells and tissue of an affected individual. Thus, the composition decrease cytotoxicity and thereby ameliorate the symptoms of cell-mediated autoimmune disorders.

The T regs can also down regulate β-cells thereby inhibiting the production of antibodies such as autoantibodies.

This strategy is unlike almost all other treatment modalities currently in use which are either anti-inflammatory or immunosuppressive. Commonly used corticosteroids suppress cytokine production and block the terminal events which cause tissue injury, but generally do not alter the underlying autoimmune response. Cytotoxic drugs or experimental genetically engineered biologicals such as monoclonal antibodies may also deplete specific lymphocyte populations or interfere with their function. These drugs are generally only moderately successful and have severe adverse side effects. Certain cytokines have been given systemically to patients, but these agents also have broad actions with associated serious adverse side effects.

By contrast, the strategy of the present invention is to produce remission by restoring normal regulatory cell function and, thus, “resetting” the immune system using T regs made according to the disclosure herein. Another significant potential advantage of this strategy is a low probability of serious adverse side effects. Since only trace amounts of regulatory compositions such as cytokines will be returned to the patient, there should be minimal toxicity.

Circulating B lymphocytes spontaneously secreting IgG are increased in patients with active SLE (Blaese, R. M., et al. (1980), Am J Med 69:345-350; Klinman, D. M. et al. (1991) Arthritis Rheum 34: 1404-1410). Sustained production of polyclonal IgG and autoantibodies in vitro requires T cell help (Shivakumar, S. et al. (1989), J Immunol 143:103-112). Previous studies of T cell regulation of spontaneous IgG production shows that while CD8+ T cells inhibit antibody production in healthy individuals, in SLE these cells support B cell function instead (Linker-Israeli, M. et al. (1990), Arthritis Rheum 33:1216-1225). In other autoimmune diseases such as rheumatoid arthritis and multiple sclerosis, T cells rather than antibody are responsible for tissue injury and the resulting inflammation (Panayi GS, et al. Arthritis Rheum (1992) 35:725-773), Allegretta M et al. Science (1990) 247:718-722.

Accordingly, in a preferred embodiment, the present invention is drawn to methods of treating antibody- and T cell-mediated autoimmune diseases that comprise removing peripheral blood mononuclear cells (PBMCs) from the patient with the autoimmune disease and treating certain of these cells with two different regulatory compositions for two sequential time periods.

Without being bound by theory, it appears there are several ways the methods of the invention may work. First of all, the treatment of the cells with the regulatory compositions leads to the direct suppression of antibody production, which can lead to amelioration of antibody-mediated autoimmune symptoms. Alternatively or additionally, the treatment of the cells induces regulatory cells to down regulate antibody production in other cells. Antibody in this context includes all forms of antibody, including IgA, IgM, IgG, IgE, etc. The net result is a decrease in the amount of antibody in the system.

Additionally, the treatment with T regs normalizes cell-mediated immune responses in patients with autoimmune diseases. Without being bound by theory, it appears that the treatment of the cells restores the balance between IL-10 and TNF-α leading to an enhanced production of Th1 cytokines and normalization of cell mediated immunity.

Furthermore, stimulation of immune cells with two different regulatory compositions suppresses cell-mediated immune responses. Without being bound by theory, it appears that CD4+ T cells can be stimulated to produce immunosuppressive levels of active TGF-β, that then suppresses harmful T and B cells. Alternatively, CD4+ T cells can be stimulated to suppress the activation and/or effector functions of other T cells by a contact-dependent mechanism of action. These effects require CD4+ cells to be activated in the presence of TGF-β.

Thus, the present invention inhibits aberrant immune responses. In patients with antibody-mediated autoimmune disorders, the present invention restores the capacity of peripheral blood T cells to down regulate antibody production and restores cell mediated immune responses by treating them with an regulatory composition ex vivo. In patients with cell-mediated disorders, the present invention generates regulatory T cells which suppress cytotoxic T cell activity in other T cells.

By “immune response” herein is meant host responses to foreign or self antigens. By “aberrant immune responses” herein is meant the failure of the immune system to distinguish self from non-self or the failure to respond to foreign antigens. In other words, aberrant immune responses are inappropriately regulated immune responses that lead to patient symptoms. By “inappropriately regulated” herein is meant inappropriately induced, inappropriately suppressed and/or non-responsiveness. Aberrant immune responses include, but are not limited to, tissue injury and inflammation caused by the production of antibodies to an organism's own tissue, impaired production of IL-2, TNF-α and IFN-γ and tissue damage caused by cytotoxic or non-cytotoxic mechanisms of action.

Accordingly, in a preferred embodiment, the present invention provides methods of treating antibody-mediated autoimmune disorders in a patient. By “antibody-mediated autoimmune diseases” herein is meant a disease in which individuals develop antibodies to constituents of their own cells or tissues. Antibody-mediated autoimmune diseases include, but are not limited to, systemic lupus erythematosus (SLE), pemphigus vulgaris, myasthenia gravis, hemolytic anemia, thrombocytopenia purpura, Grave's disease, dermatomyositis and Sjogren's disease. The preferred autoimmune disease for treatment using the methods of the invention is SLE.

In addition, patients with antibody-mediated disorders frequently have defects in cell-mediated immune responses. By “defects in cell mediated immune response” herein is meant impaired host defense against infection. Impaired host defense against infection includes, but is not limited to, impaired delayed hypersensitivity, impaired T cell cytotoxicity and impaired production of TGF-β. Other defects, include, but are not limited to, increased production of IL-10 and decreased production of IL-2, TNF-α and IFN-γ.

In a preferred embodiment, the present invention provides methods of treating cell-mediated autoimmune disorders in a patient. By “cell-mediated autoimmune diseases” herein is meant a disease in which the cells of an individual are activated or stimulated to become cytotoxic and attack their own cells or tissues. Alternatively, the autoimmune cells of the individual may stimulate other cells to cause tissue damage by cytotoxic or non-cytotoxic mechanisms of action. Cell-mediated autoimmune diseases include, but are not limited to, Hashimoto's disease, polymyositis, disease inflammatory bowel disease, multiple sclerosis, diabetes mellitus, rheumatoid arthritis, and scleroderma.

By “treating” an autoimmune disorder herein is meant that at least one symptom of the autoimmune disorder is ameliorated by the methods outlined herein. This may be evaluated in a number of ways, including both objective and subjective factors on the part of the patient. For example, immunological manifestations of disease can be evaluated; for example, the level of spontaneous antibody and autoantibody production, particularly IgG production in the case of SLE, is reduced. Total antibody levels may be measured, or autoantibodies, including, but not limited to, anti-double-stranded DNA (ds DNA) antibodies, anti-nucleoprotein antibodies, anti-Sm, anti-Rho, and anti-La. Cytotoxic activity can be evaluated as outlined herein. Physical symptoms may be altered, such as the disappearance or reduction in a rash in SLE. Renal function tests may be performed to determine alterations; laboratory evidence of tissue damage relating to inflammation may be evaluated. Decreased levels of circulating immune complexes and levels of serum complement are further evidence of improvement. In the case of SLE, a lessening of anemia may be seen. The ability to decrease a patient's otherwise required drugs such as immunosuppressives can also be an indication of successful treatment. Other evaluations of successful treatment will be apparent to those of skill in the art of the particular autoimmune disease.

By “patient” herein is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

The methods provide for the removal of blood cells from a patient. In general, peripheral blood mononuclear cells (PBMCs) are taken from a patient using standard techniques. By “peripheral blood mononuclear cells” or “PBMCs” herein is meant lymphocytes (including T-cells, B-cells, NK cells, etc.) and monocytes. As outlined more fully below, it appears that in one embodiment, the main effect of the regulatory compositions is to enable CD8+ or CD4+ T lymphocytes to suppress harmful autoimmune responses. Accordingly, the PBMC population should comprise CD8+ T cells. Preferably, only PBMCs are taken, either leaving or returning substantially all of the red blood cells and polymorphonuclear leukocytes to the patient. This is done as is known in the art, for example using leukophoresis techniques. In general, a 5 to 7 liter leukophoresis step is done, which essentially removes PBMCs from a patient, returning the remaining blood components. Collection of the cell sample is preferably done in the presence of an anticoagulant such as heparin, as is known in the art.

In some embodiments, a leukophoresis step is not required.

In general, the sample comprising the PBMCs can be pretreated in a wide variety of ways. Generally, once collected, the cells can be additionally concentrated, if this was not done simultaneously with collection or to further purify and/or concentrate the cells. The cells may be washed, counted, and resuspended in buffer.

The PBMCs are generally concentrated for treatment, using standard techniques in the art. In a preferred embodiment, the leukophoresis collection step results a concentrated sample of PBMCs, in a sterile leukopak, that may contain reagents and/or doses of the regulatory composition, as is more fully outlined below. Generally, an additional concentration/purification step is done, such as Ficoll-Hypaque density gradient centrifugation as is known in the art.

In a preferred embodiment, the PBMCs are then washed to remove serum proteins and soluble blood components, such as autoantibodies, inhibitors, etc., using techniques well known in the art. Generally, this involves addition of physiological media or buffer, followed by centrifugation. This may be repeated as necessary. They can be resuspended in physiological media, preferably AIM-V serum free medium (Life Technologies) (since serum contains significant amounts of inhibitors) although buffers such as Hanks balanced salt solution (HBBS) or physiological buffered saline (PBS) can also be used.

Generally, the cells are then counted; in general from 1×10⁹ to 2×10⁹ white blood cells are collected from a 5-7 liter leukophoresis step. These cells are brought up roughly 200 mls of buffer or media.

In one embodiment, the PBMCs may be enriched for one or more cell types. For example, the PBMCs may be enriched for CD8+ T cells or CD4+ T cells. This can be done as described in Greg, et al., (1988) J Immunol. 160:2248. In a preferred embodiment, the PBMCs are separated in a automated, closed system such as The CliniMACS System (Miltenyi Biotech) with MACS microbeads. In brief, the PBMC are washed and resuspended in CliniMACS PBS/EDTA (Miltenyi Biotech) supplemented with 2% human serum in a cell preparation bag to which antibodies contained in a CD4+ cell isolation kit (#130-091-155) or CD8+ cell isolation kit (#130-091-154) Miltenyi Biotech) are added. The cells are incubated for 30 minutes at room temperature on an orbital shaker. Cells were washed, resuspended, and applied to the CliniMACS^{PLUS} instrument. Upon completion of the depletion program, the purified T cells collected in collection bags. Generally, this is done to maintain sterility and to insure standardization of the methodology used for cell separation, activation and development of suppressor cell function.

Once the cells have undergone any necessary pretreatment, the cells are treated with first and second regulatory compositions or a first regulatory composition followed by treatment with nutrient media. By “treated” herein is meant that the cells are incubated sequentially with the first, second regulatory compositions or nutrient each for a predetermined time period sufficient to form T regs having the capacity to inhibit immune responses, including antibody and autoantibody production, particularly when transferred back to the patient. The incubation will generally be under physiological temperature. As noted above, this may happen as a result of direct suppression of antibody production by the treated cells, or by inducing regulatory cells to down regulate the production of antibody in the patient's lymphoid organs.

By first “regulatory composition” herein is meant a composition comprising TGF-β and optionally a mitogen and/or cytokine. The second “regulatory composition” comprises a cytokine, preferably IL-2.

TFG-β is a component in the first regulatory composition. By “transforming growth factor -β” or “TGF-β” herein is meant any one of the family of the TGF-βs, including the three isoforms TGF-β1, TGF-β2, and TGF-β3; see Massague, J. (1980), J Ann. Rev. Cell Biol 6:597. Lymphocytes and monocytes produce the β1 isoform of this cytokine (Kehrl, J. H. et al. (1991), Int J Cell Cloning 9: 438-450). The TFG-β can be any form of TFG-β that is active on the mammalian cells being treated. In humans, recombinant TFG-β is currently preferred. A preferred human TGF-β can be purchased from Genzyme Pharmaceuticals, Farmington, MA. In general, the concentration of TGF-β used ranges from about 2 of cell suspension to about 5 nanograms, with from about 10 pg to about 4 ng being preferred, and from about 100 pg to about 2 ng being especially preferred, and 1 ng/ml being ideal.

Suitable mitogens include, but are not limited to, T cell activators such as anti-CD2, including anti-CD2 antibodies and the CD2 ligand, LFA-3, and mixtures or combinations of T cell activators such as Concanavalin A (Con A), staphylococcus enterotoxin B (SEB), anti-CD3, anti-CD28. Anti-CD2 antibodies are known (OKT11, American Type Culture Collection, Rockville MD and GT2, Huets, et al., (1986) J Immunol. 137:1420).

When a mitogen is used, it is generally used as is known in the art, at concentrations ranging from 1 μg/ml to about 10 μg/ml is used. In addition, it may be desirable to wash the cells with components to remove the mitogen, such as α-methyl mannoside, as is known in the art.

In a preferred embodiment, T cells are strongly stimulated with mitogens, such as anti-CD2, anti-CD3, anti-CD28 or combinations of monoclonal antibodies, e.g., anti-CD3 and anti-CD28 or a specific autoantigen, if known. The presence of TGF-β in the first regulatory composition induces T cells to develop potent suppressive activity. Repeated stimulation of the T cells with our without TGF-β in secondary cultures may be necessary to develop maximal suppressive activity.

In a preferred embodiment, IL-2 is the cytokine used in the second regulatory composition. The IL-2 can be any form of IL-2 that is active on the mammalian cells being treated. In humans, recombinant IL-2 is currently preferred. Recombinant human IL-2 can be purchased from R&D Systems, Minneapolis, Minn. In general, the concentration of IL-2 used ranges from about 1 Unit/ml of cell suspension to about 100 U/ml, with from about 5 U/ml to about 25 U/ml being preferred, and with 10 U/ml being especially preferred. In a preferred embodiment, IL-2 is not used alone.

In a preferred embodiment, the invention provides methods comprising conditioning T cells, including, but not limited to CD8+ T or CD4+ T cells, and other minor T cell subsets such as CD8⁻CD4⁻, NK T cells, etc., first with TGF-β and mitogen and then with IL-2. These T cells prevent other T cells from becoming cytotoxic effector cells. In a preferred embodiment, the invention provides methods comprising conditioning CD4+ or CD8+ T cells with TGF-β to produce immunosuppresive levels of TGF-β.

In a preferred embodiment, the invention provides methods comprising conditioning CD4+ or CD8+ T cells first with TGF-β and mitogen and then with IL-2 to produce T cells that suppress by a contact-dependent mechanism.

The regulatory composition is incubated with the cells for a period of time sufficient to cause an effect. In a preferred embodiment, treatment of the cells with the regulatory composition is followed by immediate transplantation back into the patient. Accordingly, in a preferred embodiment, the cells are incubated with the regulatory composition for 12 hours to about 7 days. The time will vary with the suppressive activity desired. For suppression of antibody production 48 hours is especially preferred and 5 is especially preferred for suppression of cytotoxicity.

In one embodiment, the cells are treated for a period of time, washed to remove the regulatory composition, and may be reincubated to expand the cells. Before introduction into the patient, the cells are preferably washed as outlined herein to remove the regulatory composition. Further incubations for testing or evaluation may also be done, ranging in time from a few hours to several days. If evaluation of antibody production prior to introduction to a patient is desirable, the cells will be incubated for several days to allow antibody production (or lack thereof) to occur.

Once the cells have been treated, they may be evaluated or tested prior to autotransplantation back into the patient. For example, a sample may be removed to do: sterility testing; gram staining, microbiological studies; LAL studies; mycoplasma studies; flow cytometry to identify cell types; functional studies, etc. Similarly, these and other lymphocyte studies may be done both before and after treatment.

In a preferred embodiment, the quantity or quality, i.e. type, of antibody production, may be evaluated. Thus, for example, total levels of antibody may be evaluated, or levels of specific types of antibodies, for example, IgA, IgG, IgM, anti-DNA autoantibodies, anti-nucleoprotein (NP) antibodies, etc. may be evaluated. Regulatory T cells may also be assessed for their ability to suppress T cell activation or to prevent T cell cytotoxicity against specific target cells in vitro.

In a preferred embodiment, the levels of antibody, particularly IgG, are tested using well known techniques, including ELISA assays, as described in Abo et al. (1987), Clin. Exp. Immunol. 67:544 and Linker-Israeli et al. (1990), Arthritis Rheum 33:1216, both of which are hereby expressly incorporated by reference. These techniques may also be used to detect the levels of specific antibodies, such as autoantibodies.

In a preferred embodiment, the treatment results in a significant decrease in the amount of IgG and autoantibodies produced, with a decrease of at least 10% being preferred, at least 25% being especially preferred, and at least 50% being particularly preferred. In many embodiments, decreases of 75% or greater are seen.

In a preferred embodiment, prior to transplantation, the amount of total or active TGF-β can also be tested. As noted herein, TGF-β is made as a latent precursor that is activated post-translationally.

After the treatment, the cells are transplanted or reintroduced back into the patient. This is generally done as is known in the art, and usually comprises injecting or introducing the treated cells back into the patient, via intravenous administration, as will be appreciated by those in the art. For example, the cells may be placed in a 50 ml Fenwall infusion bag by injection using sterile syringes or other sterile transfer mechanisms. The cells can then be immediately infused via IV administration over a period of time, such as 15 minutes, into a free flow IV line into the patient. In some embodiments, additional reagents such as buffers or salts may be added as well.

After reintroducing the cells into the patient, the effect of the treatment may be evaluated, if desired, as is generally outlined above. Thus, evaluating immunological manifestations of the disease may be done; for example the titers of total antibody or of specific immunoglobulins, renal function tests, tissue damage evaluation, etc. may be done. Tests of T cells function such as T cell numbers, phenotype, activation state and ability to respond to antigens and/or mitogens also may be done.

The treatment may be repeated as needed or required. For example, the treatment may be done once a week for a period of weeks, or multiple times a week for a period of time, for example 3-5 times over a two week period. Generally, the amelioration of the autoimmune disease symptoms persists for some period of time, preferably at least months. Over time, the patient may experience a relapse of symptoms, at which point the treatments may be repeated.

In a preferred embodiment, the invention further provides kits for the practice of the methods of the invention, i.e., the incubation of the cells with the regulatory compositions. The kit may have a number of components. The kit comprises a cell treatment container that is adapted to receive cells from a patient with an antibody-mediated or cell-mediated autoimmune disorder. The container should be sterile. In some embodiments, the cell treatment container is used for collection of the cells, for example it is adaptable to be hooked up to a leukophoresis machine using an inlet port. In other embodiments, a separate cell collection container may be used.

In a preferred embodiment, the kit comprises a cell treatment container that is adapted to receive cells from a patient with a cell mediated disorder. The kit may also be adapted for use in a automated closed system to purify specific T cell subsets and expand them for transfer back to the patient.

The form and composition of the cell treatment container may vary, as will be appreciated by those in the art. Generally the container may be in a number of different forms, including a flexible bag, similar to an IV bag, or a rigid container similar to a cell culture vessel. It may be configured to allow stirring. Generally, the composition of the container will be any suitable, biologically inert material, such as glass or plastic, including polypropylene, polyethylene, etc. The cell treatment container may have one or more inlet or outlet ports, for the introduction or removal of cells, reagents, regulatory compositions, etc. For example, the container may comprise a sampling port for the removal of a fraction of the cells for analysis prior to reintroduction into the patient. Similarly, the container may comprise an exit port to allow introduction of the cells into the patient; for example, the container may comprise an adapter for attachment to an IV setup.

The kit further comprises at least one dose of first regulatory composition comprising TGFβ and optionally one or more cytokines or mitogens. The components may be separate doses or combined. For example, TGFβ can be combined with at least one or more cytokines and/or one or more mitogens. In preferred embodiments, the mitogens are immunobeads coated with anti-CD3 antibody, or anti-CD28 antibody or immunobeads coated with a combination of anti-CD3 and anti-CD-28 antibodies or anti-CD3 in combination with anti-CD28 antibody. The kit may also contain at least one dose of a second regulatory composition containing one or more cytokines such as IL-2, IL-7, IL-10 and/or IL-15 with IL-2 being preferred. The kit may also contain at least one dose of nutrient media for diluting the first culture and/or to dissolve lyophilized kit components. “Dose” in this context means an amount of the regulatory composition that is sufficient to cause an effect. In some cases, multiple doses may be included. In one embodiment, the dose may be added to the cell treatment container using a port; alternatively, in a preferred embodiment, the first regulatory composition is already present in the cell treatment container. In a preferred embodiment, the regulatory compositions and/or nutrient media are lyophilized for stability, and are reconstituted using nutrient media, or other reagents.

In some embodiments, the kit may additionally comprise at least one reagent, including buffers, salts, media, proteins, drugs, etc. For example, mitogens, monoclonal antibodies and treated magnetic beads for cell separation can be included. In some embodiments, the kit may additionally comprise written instructions for using the kits.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

Purified T cells from DBA/2 mice were stimulated with beads coated with anti-CD3 and anti-CD28, with IL-2 and TGF-β1 2 ng/ml (T reg) or without TGFβ1 (T con) for 6 days. To induce a lupus-like syndrome, 80 million D2 spleen cells were transferred to DBA/2×C57BL/6F1 mice. Some groups also received 5 to 20 million T con or T reg (n=3). Two weeks later, the mice were bled and assessed for anti-dsDNA antibody and total IgG that were measured by standard ELISA methods. See FIGS. 1A and 1B.

These results demonstrate that T cells continuously stimulated with anti-CD3/CD28 antibodies in the presence of TGF-β failed to develop suppressive activity in vivo.

### Example 2

Since continuous anti-CD3 and anti-CD28 polyclonal stimulation in the presence of TGF-β failed to induce T cells to develop suppressor activity, the timing of the primary culture from continuous stimulation for 6 was modified to 2 days followed by culturing these cells 4 more days before harvesting (2+4 protocol).

Purified T cells from DBA/2 mice were stimulated with beads coated with anti-CD3 and anti-CD28 with IL-2 and TGF-β1 2 ng/ml, (T reg) or without TGF-β1 (T con) for 2 days. The beads were then removed and the cells cultured for another 4 days in the presence of IL-2 (10 u/ml). The CD4⁺CD25⁺ cells or CD4⁺CD25⁻ cells were sorted from the T reg or T con cells and the FoxP3 mRNA expression was determined on various cell subsets by RT-PCR. See FIG. 2.

### Example 3

CD4⁺CD25⁻ cells were stimulated with anti-CD3/CD28 coated beads (1:3)+IL-2 (40 u/ml) for 6 days +/−TGF-β(2 ng/ml). In some groups, these cells were stimulated with similar beads for 2 days, then beads were removed and the cells cultured for another 4 days with 10 u/ml IL-2. FoxP3 mRNA was analyzed by RT-PCR (left) or semiquantitative levels were determined by normalization to HPRT of three separate experiments (right). M indicates medium only, T indicates TGF-β. See FIG. 3.

These results indicate that CD4 cells activated with TGF-β using the 2 day plus 4 day protocol to produce a polyclonal population that express much more FoxP3 than CD4 cells that were continuously stimulated.

### Example 4

T cells were labeled with CFSE and stimulated with soluble anti-CD3 in the presence of APC for 4 days. CD4⁺CD25⁺ cells were sorted as described before and various ratio of these cells were added to culture. The CD4⁺ cell proliferation was analyzed by the dilution of CFSE. See FIG. 4.

These results demonstrate that T cells activated with TGF-β for 2 days and cultured for 4 days with IL-2 became CD4⁺CD25⁺ suppressor cells.

### Example 5

CD4⁺ cells were briefly activated with a polyclonal mitogen and TGF-β and cultured for 4 more days. CD4⁺ positive cells became CD25⁺ cells with marked suppressor activity. See FIG. 5.

### Example 6

Purified T cells from DBA/2 mice were stimulated with beads coated with anti-CD3 and anti-CD28 with IL-2 and TGF-β1 2 ng/ml (T reg) or without (T con) for 2 days and remove beads and cultured for 4 days. To induce a lupus-like syndrome, 80 million D2 spleen cells were transferred to DBA/2×C57BL/6 F1 mice. Some groups also received 5 to 20 million T con or T reg (n=3). Two weeks later, the mice were bled and assessed for anti-dsDNA antibody and total IgG that were measured by standard ELISA methods. See FIG. 6.

These results demonstrate that CD4⁺ cells polyclonally activated with TGF-β using the 2+4 protocol were induced to develop a strong suppressive activity in vivo.

### Example 7

Naive human CD4⁺ cells were stimulated with anti-CD3/CD28 beads in the presence or absence of TGF beta. The cells in some wells were cultured for 6 days (continuous). At day 2 of culture, the cells from some wells were removed, distributed into two wells, and fresh medium added to make a final volume of 1 ml. These were recultured for 4 additional days (2+4). At this time the cells were assayed for expression of FoxP3. Data represent FoxP3+ cells as a percentage of CD25+ cells.

These results indicate that to induce CD4 regulatory T cells with TGF-β, the 2 +4 protocol yields a higher percentage as well as total numbers (not shown) of FoxP3+ cells.

### Example 8

Naive CD4+ cells were stimulated with anti-CD3/CD28 beads in the presence or adsence of TGF beta. At day 2 of culture, the cells were removed from the wells, and split into two portions. The magnetic beads were removed from one portion. Each portion was added to new wells with additional medium to a final volume of 1 ml and the cells were recultured for 4 additional days. At this time the cells were assayed for expression of FoxP3. Data represent the % FoxP3+ cells as a percentage of CD25+ cells.

These results reveal that naive CD4+ cells require continuous stimulation for optimal expression of the transcription factor, FoxP3.

What is claimed is:

1. A method for making regulatory T cells comprising:
culturing peripheral blood mononuclear cells (PBMC) with a first regulatory composition comprising TGF-β and optionally a mitogen and/or cytokine;
removing said first regulatory composition from said PBMC cells; and
culturing said PBMC cells with (a) a second regulatory composition or (b) nutrient medium.

2. The method of claim 1 wherein said first regulatory composition further comprises a cytokine.

3. The method of claim 1 wherein said first regulatory composition further comprises a mitogen.

4. The method of claim 3 wherein said mitogen comprises anti-CD3 and/or anti-CD28 antibodies.

5. The method of claim 4 wherein said second regulatory composition is substantially free of cytokine.

6. The method of claim 4 wherein said second regulatory composition is substantially free of IL-2

7. The method of claim 1 wherein said mitogen is selected from the group consisting of anti-CD2, and anti-CD3, anti-CD28 antibodies and combinations thereof.

8. The method of claim 7 wherein said mitogen comprises anti-CD3 antibody.

9. The method of claim 7 wherein said mitogen comprises anti-CD3 and anti-CD28 antibodies.

10. The method of claim 1 wherein said cytokine comprises IL-2.

11. The method of claim 1 wherein said culturing with said first regulatory composition is for 24-48 hours and said culturing with said second regulatory composition is for 4-6 days.

12. The method of claim 1 wherein said mitogen is anti-CD3 or anti-CD3 in combination with anti-CD28 and said culturing with said mitogen and TGF-β is for 24-48 hours and wherein said cytokine is IL-2 and said contacting with said second regulatory composition is for 4-6 days.

13. The method of claim 1 wherein the concentration of said mitogen is from 0.2 to 20 g/ml.

14. The method of claim 1 or 12 wherein said mitogen is linked to beads wherein there are between 1:10 beads per PBMC cell and 1:1 beads per PBMC cell and the concentration of said cytokine is between 2 units and 50 units per ml.

15. The method of claim 1 wherein said PBMCs comprise CD4⁺ and/or CD8⁺ cells.

16. The method of claim 1 wherein said PBMCs comprise NK-T cells.

17. A method for making regulatory T cells comprising:
culturing a population of peripheral blood mononuclear cells (PBMCs) with a first regulatory composition comprising TGF-β and optionally a mitogen and/or cytokine for a first time period to form a first culture;
diluting said first culture with nutrient medium to form a second culture of PBMCs; and
culturing said second culture to form said regulatory T cells.

18. The method of claim 17 wherein nutrient medium is substantially free of TGF-β.

19. The method of claim 17 wherein said nutrient medium comprises at least one cytokine.

20. The method of claim 19 wherein said cytokine comprises IL-2, IL-7, IL-10 and/or IL-15.

21. The method of claim 17 wherein said nutrient medium comprises anti-CD3 and/or anti-CD-28.

22. The method of claim 17 wherein said nutrient medium comprises beads coated with anti-CD3 and/or anti-CD28 antibody.

23. The method of claim 17 wherein said diluting comprises dividing said first culture into two or more portions and adding nutrient medium to said portions.

24. The method of claim 23 wherein the PBMCs of said first culture form cell clusters during said culturing and said dividing of said first culture causes a mechanical breakdown in the size of said cell clusters in said second culture.

25. The method of claim 24 wherein the breakdown in the size of said cell clusters results in the enhanced production of regulatory T-cells during the culturing of said second culture as compared to when said second culture is not divided.

26. Regulatory T cells made according to the method of claim 1, 12 or 17.

27. A method for treating an autoimmune disorder in a patient comprising removing peripheral blood mononuclear cells (PBMC) from said patient; treating said PBMC cells according to claim 1 or 18 for forming regulatory T cells; and introducing said regulatory T cells to said patient.

28. A method for treating an autoimmune disorder in a patient comprising removing peripheral blood mononuclear cells (PBMC) from said patient; treating said PBMC cells with anti-CD3 antibody and TGF-β to form regulatory T cells and introducing said regulatory T cells into said patient.
